(19) United States
(12) Patent Application Publication
(10) Pub. No.: US 2007/0055093 A1
(43) Pub. Date: Mar. 8, 2007

(54) Implantable warp knitted fabric

(76) Inventor: Jean-Marc Beraud, Saint- Etienne (FR)

Correspondence Address: CLARK & BRODY, 1090 VERMONT AVENUE, NW, SUITE 250, WASHINGTON, DC, 20005, US

(21) Appl. No.: 11/220,673

(22) Filed: Sep. 8, 2005

(51) Int. Cl. A61F 2/02

(52) U.S. Cl. 600/30

(57) ABSTRACT

A warp knit designed for surgical use in biocompatible yarn is characterized in that it has a woven chain stitch. The warp knit can be used in implantation to ensure the reinforcement of muscular regions and in the production of parietal reinforcement prostheses or tapes to treat stress incontinence.

### FIELD OF THE INVENTION

The present invention concerns the technical area of fabrics or textile cloth for surgical use intended for implantation to ensure the reinforcement of muscular regions, soft tissues or further the walls of inner organs of the human body.

### BACKGROUND OF THE INVENTION

In the above sphere, it is known to use biocompatible materials in non-woven fibres or crossed-thread fabrics which are cut to size by the surgeon.

While said sheets of textile materials indeed provide flexible, implantable reinforcements they nonetheless have some drawbacks. Firstly, as they have to be cut to size the reinforcement pieces obtained have edges which may injure the tissues in the implant zone.

Also, having regard to their structure said reinforcement pieces are liable to release free particles which may effect the healing of the implant tissues. In addition, in some uses such as tapes to treat urinary stress incontinence, these tapes have the disadvantage of deforming and rolling up whenever a longitudinal force or traction is exerted upon them.

Similarly, said tapes cut in crossed-thread fabrics have a certain extent of elasticity which make them unfit for use with implants for residual tension-free insertion as is particularly sought in new treatment protocols for urinary stress incontinence.

To overcome this disadvantage patent application WO 02 065 944 proposed a warp knit for surgical use made in biocompatible yarn and having free loops on its longitudinal edges intended to form implant elements.

The knit in application WO 02 065 944 then has a simple one-bar tricot weave or atlas-type weave that is too susceptible to running, which means that its characteristics may be affected if the tape happens to be damaged by the surgeon during implant procedure.

U.S. Pat. No. 5,456,711 also proposed a warp knit for surgical use in compatible materials having a two-bar weave more commonly known as Queenscord. This knit has the feature of offering better resistance to running than the previous knit but nevertheless has the disadvantage of not offering porosity able to promote a rapid healing process.

### BRIEF SUMMARY OF THE INVENTION

There is therefore an apparent need for a new type of knit adapted for implantation under so-called tension-free procedures to treat cystocele or female urinary stress incontinence, the knit being run-proof and also particularly benefiting rapid fibroplastic ingrowth to promote optimum pain-free healing.

To reach this objective the invention concerns a warp knit for surgical use in biocompatible yarn characterized in that its weave contains a woven chain stitch.

It has been found that said woven chain stitch offers an openwork factor and pores of sufficient dimensions to promote fibroplastic ingrowth. Also said woven chain stitch particularly promotes the obtaining of a locked knit that is almost run-proof and is therefore resistant against cuts accidentally or purposefully made by the surgeon at the time of insertion.

According to one characteristic of the invention the warp knit is made so that it has a closed chain stitch sequence of 1-0 type or accessorily an open chain stitch sequence of 1-0/0-1 type and an underlap sequence of 0-0/3-3 type.

According to another characteristic of the invention, the knit is made so that it has a closed chain stitch sequence of 1-0 type and an underlap sequence of 1-0/2-3 type which reinforces the run-proof nature of the knit.

A warp knit of the invention may be made on any type of suitable two-bar knitting loom such as RASCHEL looms or CROCHET looms.

When implementing on a loom with two guide bars, one of the bars ensures the chain stitch sequence whilst the other bar ensures the underlap sequence.

For particular fostering of the fibroplastic ingrowth mechanism, the warp knit of the invention is made so that the pore size in at least one direction is greater than 74 μm and preferably, but not necessarily, of any dimension lying between 20 μm and 80 μm.

Preferably the warp knit of the invention is made in ribbon form with smooth edges, and its width is preferably but not necessarily between 8 mm and 55 mm and further preferably between 8 mm and 15 mm. Said embodiment of the warp knit of the invention makes it particularly suitable for forming a reinforcement tape to treat cystocele or urinary stress incontinence, comprising at least one length of the knit of the invention.

According to a further characteristic of the invention, again to achieve optimum healing results, the warp knit of the invention is made so that its unit weight lies between 28 g/m² and 100 g/m² and further preferably of between 30 g/m² and 40 g/m².

In accordance with the invention the warp knit for surgical use may be made in yarns of biocompatible synthetic material such as polyamide, polyoxymethylene, polyetherketone, polypropylene, polyethylene or polyester yarn. Preferably, but not strictly necessarily, the yarns forming the warp knit of the invention are chosen from among polypropylene, polyester and polyamide. It is to be noted that in a particularly preferred embodiment, the warp knit of the invention is made in polypropylene yarn.

Different types of thread may be used to produce a warp knit of the invention, such as multi-filament thread or mono-filament thread. Preferably monofilament thread is chosen on account of its low propensity to release particles.

Therefore in a preferred embodiment the warp knit of the invention is knitted using mono-filament yarn whose diameter lies between 0.017 mm and 0.15 mm, preferably between 0.05 mm and 0.10 mm.

According to a further characteristic of the invention, in order to provide extensive dimensional stability the warp knit for surgical use of the invention is given a heat-setting finish at a temperature of between 120° C. and 140° C., preferably between 125° C. and 135° C.

Said heat-setting may be conducted by vaporizing or by calendaring.

The invention also concerns the use of a knit of the invention to produce an implantable prosthesis, and preferably the use of a knit of the invention in the form of a tape with smooth edges.

The invention also concerns the use of a knit of the invention to ensure the reinforcement of an inner region of the human body under surgical implant procedure.

Various other characteristic of the invention will become apparent from the description given below with reference to the appended drawings which illustrate preferred forms of embodiment of a warp knit for surgical use according to the invention.

### BRIEF DESCRIPTION OF THE DRAWINGS

FIG. 1 is a photo illustrating a first embodiment of a warp knit of the invention,

FIG. 2 is a detailed view illustrating the interlacing of the stitches of the warp knit of the invention,

FIG. 3 is an optical microscope view of the warp knit such as illustrated FIG. 1.

FIG. 4 is a simplified diagram of the structure of the warp knit such as illustrated FIGS. 1 and 2,

FIG. 5 is a simplified diagram of a variant of embodiment of a warp knit of the invention.

### DETAILED DESCRIPTION OF THE INVENTION

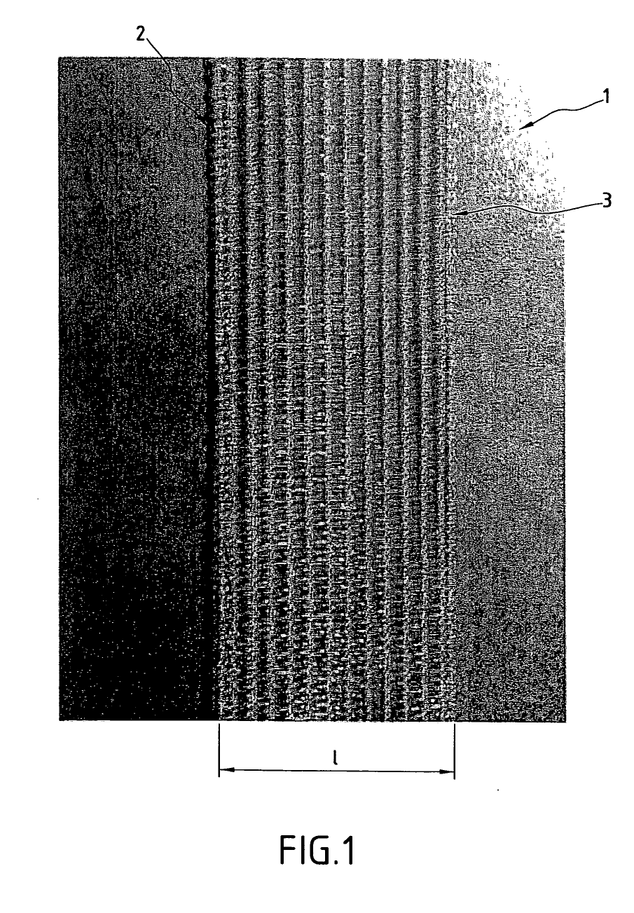

The invention sets out to propose a warp knit such as illustrated FIG. 1 and designated reference 1 particularly suitable for surgical use and in particular for the treatment protocol of cystocele by reinforcing the muscles in the pelvic floor to give support to the urethra by means of a tape.

Therefore a warp knit of the invention is made using biocompatible yarn in synthetic material such as polyamide, polyoxymethylene, polyetherketone, polypropylene, polyethylene or polyester.

According to the invention, the knit 1 may be made using any type of thread such as multi-filament thread and mono- or multi-material thread.

Preferably knit 1 of the invention is rather more made in a mono-filament yarn having a diameter of between 0.017 mm and 0.15 mm and preferably between 0.05 mm and 0.10 mm.

In a particularly preferred embodiment, knit 1 of the invention is made in polypropylene mono-filament yarn having a diameter of approximately 0.08 mm.

As shown FIG. 1 the warp knit 1 is preferably knit in the form of a ribbon or tape having smooth edges 2 and 3. Preferably, but not strictly necessarily, the knit 1 is made in the form of a tape having a width of between 8 mm and 55 mm and further preferably between 8 mm and 15 mm for use as support tapes to treat cystocele for example.

As described above, according to a preferred embodiment of the invention the edges 2 and 3 are made smooth so that no free filament is left which may harm the tissues when inserting the tape.

Knit 1 of the invention is made so that it has low elasticity and even, if possible, with no elasticity to allow tension-free insertion. To achieve this objective the knit is made on the basis of a woven chain stitch on a RASCHEL loom having at least two guide bars or even a crochet loom.

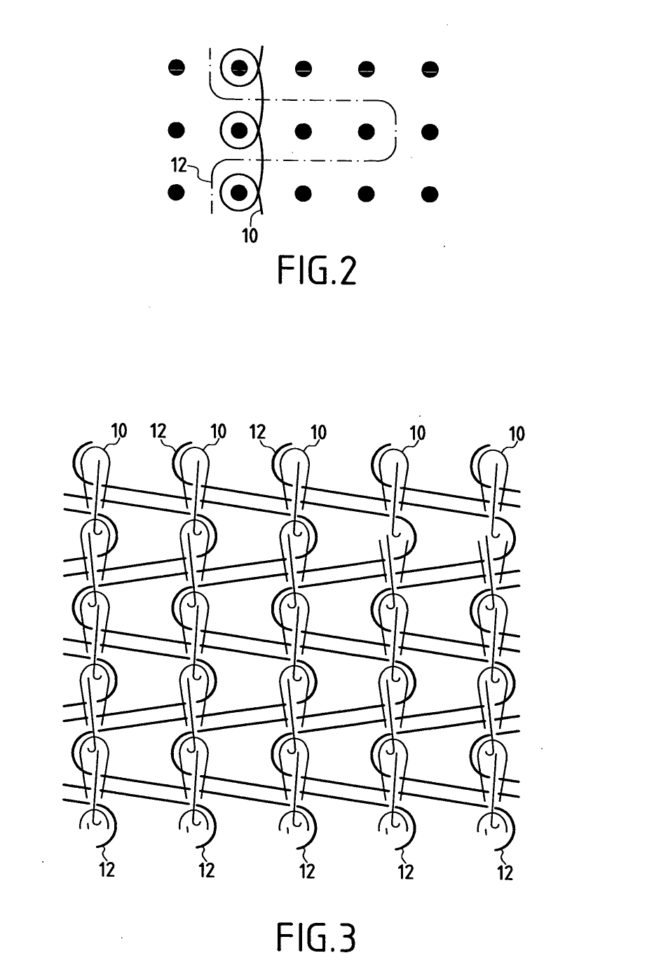

For implementation on a loom with two guide bars, the knit 1 such as illustrated FIG. 1 is made by adopting an underlap schema such as schematically shown FIG. 2. Therefore the yarn 10 represents the pattern made by the first bar corresponding to a closed chain stitch of 1-0 sequence type. Yarn 12 shown as a chain-dotted line represents the underlap made by the second guide bar according to a sequence of 0-0/3-3 type.

In a manner known to persons skilled in the art, the sequence such as expressed above represents the passes of the guide bars from one row to another, coordinate 1 corresponding to an intermediate position between the two first columns of needles on the left.

The knit obtained then has a structure as seen through a microscope as illustrated FIG. 3.

The structure, amongst other advantageous characteristics, has the property of being almost run-proof so that the surgeon is able to make several partial cuts therein without the risk of complete unravelling of the implanted tricot assembly.

In addition the structure of the woven chain stitch has the advantage of offering pores whose size, at least in one of their three dimensions, is greater than 74 μm. Therefore as in the illustrated example the main pores 15 are of rectangular shape with a width l₁₅ greater than 74 μm.

The tricot of the invention is preferably made so as to have a unit weight in the order of 35 g/m² bearing in mind that this weight may lie between 28 g/m² and 100 g/m² said weight being favourable for good implanting.

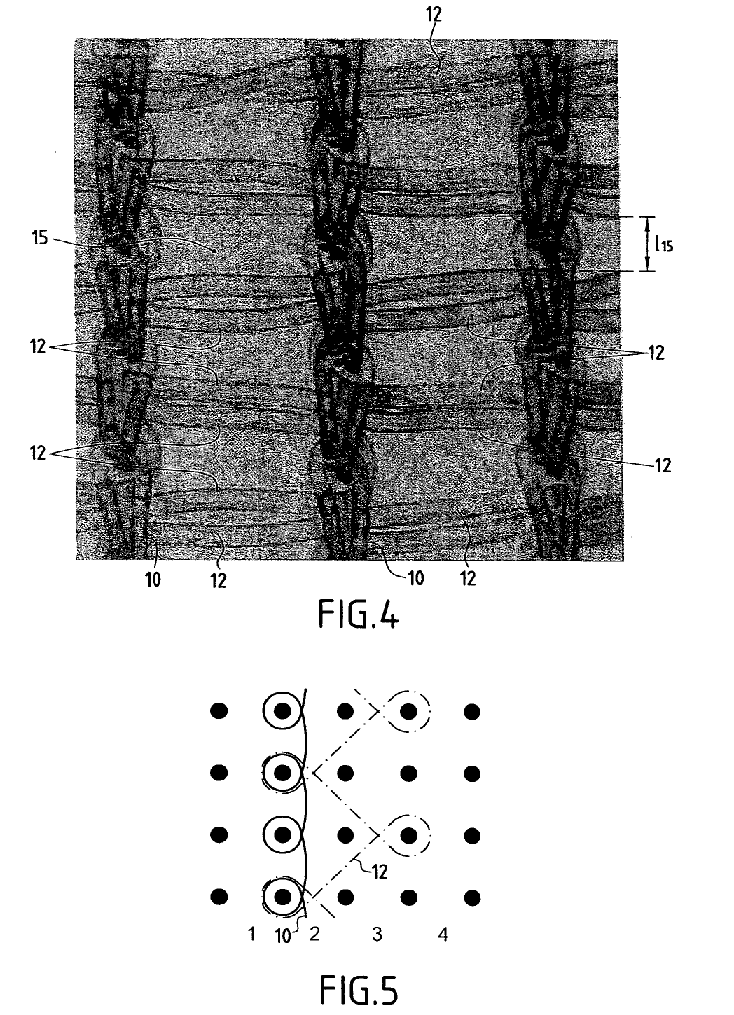

FIG. 4, in which the same references 10 and 12 have been chosen for the yarns corresponding to the two guide bars, illustrates a variant of embodiment with an open chain stitch sequence of 1-0/0-1 and an underlap sequence of 0-0/3-3 type.

In another variant of embodiment the knit is made so as to have a fully run-proof structure.

For this purpose the second guide bar taking in charge yarn 12 is given a symmetrical stitching movement with respect to the chain stitch guide bar, and gives a weave whose sequence is 1-0 for the chain stitch and 1-0/2-3 for the underlap thread 12 as illustrated schematically FIG. 5.

Evidently various other modifications may be made to the invention without departing from the scope thereof.

1. Warp knit for surgical use in biocompatible yarn, characterized in that its weave has a woven chain stitch.

2. Warp knit for surgical use as in claim 1, characterized in that it has a closed chain stitch sequence of 1-0 type, and an underlap sequence of 0-0/3-3 type.

3. Warp knit for surgical use as in claim 1, characterized in that it has a closed chain stitch sequence of 1-0 type and an underlap sequence of 1-0/2-3 type.

4. Warp knit for surgical use as in claim 1, characterized in that it is obtained on a knitting loom comprising two guide bars, one bar ensuring the chain stitch sequence and one bar ensuring the underlap sequence.

5. Warp knit for surgical use as in claim 1 characterized in that it has pores (15) whose size, at least in one dimension, is greater than 74 μm.

6. Warp knit for surgical use as in claim 1, characterized in that it is in the form of a tape with smooth edges.

7. Warp knit for surgical use as in claim 6 characterized in that it has a width (l) of between 8 mm and 55 mm preferably between 8 mm and 15 mm.

8. Warp knit for surgical use as in claim 1, characterized in that it has a unit weight of between 28 g/² and 100 g/m², preferably between 30 g/m² and 40 g/m².

9. Warp knit for surgical use as in claim 1, characterized in that it is made in yarn of synthetic material chosen from among polypropylene, polyester and polyamide.

10. Warp knit for surgical use as in claim 9 characterized in that it is made in mono-filament thread.

11. Warp knit for surgical use as in claim 10, characterized in that the mono-filament thread has a diameter of between 0.017 mm and 0.15 mm, preferably between 0.05 mm and 0.10 mm.

12. Warp knit for surgical use as in claim 1, characterized in that it has undergone a heat setting finish at a temperature of between 120° C. and 140° C., preferably between 125° C. and 135° C.

13. Reinforcement strip to treat cystocele characterized in that it comprises a knit length as in claim 1.

14. Use of a knit piece as in claim 1 to make an implantable prosthesis.
